(19) United States
(12) Patent Application Publication
(10) Pub. No.: US 2008/0269636 A1
(43) Pub. Date: Oct. 30, 2008

(54) System for and Method of Conveniently and Automatically Testing the Hearing of a Person

(75) Inventors: Mark Burrows, Princeton, NJ (US); John Cronin, Jericho, VT (US); Nancy Edwards, Jericho, VT (US); Tushar Narsana, Naperville, IL (US); Steven A. Shaya, Highlands, NJ (US); John Anthony Singarayar, Skillman, NJ (US)

Correspondence Address: MCCARTER & ENGLISH, LLP, FOUR GATEWAY CENTER, 100 MULBERRY STREET, NEWARK, NJ, 07102, US

(73) Assignee: Johnson & Johnson Consumer Companies, Inc., Skillman, NJ (US)

(21) Appl. No.: 11/570,463

(22) PCT Filed: Jun. 10, 2005
(86) PCT No.: PCT/US05/20756

§ 371 Date: Feb. 7, 2008

Related U.S. Application Data
(60) Provisional application No. 60/579,369, filed on Jun. 14, 2004.
(60) Provisional application No. 60/579,947, filed on Jun. 15, 2004.

(51) Int. Cl. A61B 5/00

(52) U.S. Cl. 600/559

(57) ABSTRACT

System and method for conducting a hearing test that is accessible to a mass market of individuals with potential hearing loss. The hearing test is stored on a centrally located computer (140) that is accessible via communications device (121). The system provides step-by-step guidance on the next steps to be taken if hearing loss is found, and provides a means to store and organize the user test data to create a means for reuse of the data.

### CROSS REFERENCE TO RELATED APPLICATION

This application claims the benefit of U.S. Provisional Application Nos. 60/579,947 filed Jun. 15, 2004 and 60/579/369 filed Jun. 14, 2004, assigned to the assignee of this application and incorporated by reference herein. The subject matter of International Application No. ______, filed Jun. 10, 2005 and entitled “LOW-COST HEARING TESTING SYSTEM AND METHOD OF COLLECTING USER INFORMATION,” assigned to the assignee of this application and incorporated by reference herein, is related to this application.

### FIELD OF THE INVENTION

The present invention relates to hearing testing systems, and more particularly, administering a hearing test on a low-cost system based on a toll-free telephone number or an Internet Web site. The inventive systems are programmed with a set of hearing test modules (frequencies at various amplitudes or with questions regarding issues that affect hearing or speech intelligibility), along with verbal or text instructions, which guide the person being tested to other hearing test modules based upon the user's response to the current hearing test module. In so doing, the user ends up in a pre-professional hearing test that then automatically guides the user to take a related action based upon the test results, for example, to seek further professional testing.

The present invention also relates to a method of collecting and storing user profile and hearing condition information. More particularly, the present invention relates to method of conveniently locating an automatic hearing testing system with a local database that captures user information and subsequently updates the user information onto a central database that can be used for marketing purposes.

### BACKGROUND OF THE INVENTION

More than 25 million Americans have hearing loss, including one out of four people older than 65. Hearing loss may come from infections, strokes, head injuries, some medicines, tumors, other medical problems, or even excessive earwax. It can also result from repeated exposure to very loud noise, such as music, power tools, or jet engines. Changes in the way the ear works as a person ages can also affect hearing.

For most people who have a hearing loss, there are ways to correct or compensate for the problem. If an individual has trouble hearing, that individual can visit a doctor or hearing health care professional to find out if he or she has a hearing loss and, if so, to determine a remedy. The U.S. Food and Drug Administration (FDA) and similar governing bodies in other countries have rules to ensure that treatments for hearing loss—medicines, hearing aids, and other medical devices—are tried and tested.

However, most people do not even know that they have a hearing loss. Typical indications that an individual has hearing loss include: (1) shouting when talking to others, (2) needing the TV or radio turned up louder than other people do, (3) often having to ask people to repeat what they say because the individual can't quite hear them, especially in groups or when there is background noise, (4) not being able to hear a noise when not facing the direction it's coming from, (5) seeming to hear better out of one ear than the other, (6) having to strain to hear, (7) hearing a persistent hissing or ringing background noise, and (8) not being able to hear a dripping faucet or the high notes of a violin. If an individual experiences one of more of the above indications, the individual should see his or her doctor or hearing health care professional for further testing for potential hearing loss.

To find out what kind of hearing loss the individual has and whether all the parts of the individual's ear are functioning, the person's doctor may want him or her to take a hearing test. A health care professional that specializes in hearing, such as an audiologist, often gives these tests. Audiologists are usually not medical doctors, but they are trained to give hearing tests and interpret the results. Hearing tests are painless.

If the hearing test shows that the individual has a hearing loss, there may be one or more ways to treat it. Possible treatments include medication, surgery, or a hearing aid. Hearing aids can usually help hearing loss that involves damage to the inner ear. This type of hearing loss is common in older people as part of the aging process. However, younger people can also have hearing loss from infections or repeated exposure to loud noises.

In a well-known method of testing hearing loss in individuals, the threshold of the individual's hearing is typically measured using a calibrated sound-stimulus-producing device and calibrated headphones. The measurement of the threshold of hearing takes place in an isolated sound room, usually a room where there is very little audible ambient noise. The sound-stimulus-producing device and the calibrated headphones used in the testing are known as an audiometer.

A professional audiologist performs a professional hearing test by using the audiometer to generate pure tones at various frequencies between 125 Hz and 12,000 Hz that are representative of a variety of frequency bands. These tones are transmitted through the headphones of the audiometer to the individual being tested. The intensity or volume of the pure tones is varied until the individual can just barely detect the presence of the tone. For each pure tone, the intensity at which the individual can just barely detect the presence of the tone is known as the individual's air conduction threshold of hearing. Although the threshold of hearing is only one element among several that characterizes an individual's hearing loss, it is the predominant measure traditionally used to acoustically fit a hearing compensation device.

Known audiometers are of two main types: the manual and the “automatic” type. In the manual system for and method of testing hearing, a skilled operator adjusts the audiometer controls, thereby sending a plurality of audio signals through either earphones, loudspeakers, or bone vibrators to a subject sitting in a quiet room. The subject is requested to signal to the operator, by activating a switch connected to a pilot light, by raising a hand, or by any other visible or audible means, whenever he or she has heard the sound being sent. The operator watches for the subject's responses, interprets them, and translates them into written information on a chart. This information is represented by a graph called an audiogram, which represents the threshold of hearing of the subject for a plurality of audio frequencies.

In the automatic method known as the Bekesy method of hearing testing, the audiometer presents automatically changing tone frequencies to the subject while the intensity of the signal is controlled by the subject by means of a pushbutton switch activating a motor controlling the motion of an intensity attenuator. The subject's responses are also automatically recorded by a writing pen moving over a chart as the test progresses. While the Bekesy method was considered by those skilled in the art of audiology to be a major advance, it still requires the presence of a skilled operator and the use of rather sophisticated mechanical systems. Since the introduction of the Bekesy method, an automatic method of hearing testing has been proposed in U.S. Pat. No. 4,107,465, that dispenses with the need for a skilled operator and the use of rather sophisticated mechanical systems.

Although the professional test is complete and allows for a thorough diagnostic, most hearing-impaired individuals are not even aware that they are in need of a hearing test, even if some of the aforementioned symptoms exist. What is required is a way to recognize early onset of hearing loss without the need to visit the audiologist.

Indeed, there are some new methods for testing hearing loss, albeit at a less professional level, such as programs available on the Internet. To use such a program, a user logs onto a free hearing test Web site, adjusts his or her computer speaker volume to a supplied test frequency, and uses a mouse to click on various hyperlinks on a Web page on which the user can listen to various tones and determine how many tones he or she is able to hear. The user then is guided to instructional and “next step” pages. There are a number of problems associated with this method. First, most people that have hearing loss are older, and the Internet may truly not be accessible because of their level of use of technology. Second, many low-income families cannot afford computers to run the Internet programs. Lastly, this system does not “pull” users to the site; an individual has to know both that he or she wants to be tested and that a site like this exists (i.e., from advertisements). No business entity could afford to mass market such a site. Therefore, even though some low-cost non-professional hearing tests are available, there exists a need for an improved means for hearing tests that is more accessible and can be driven in the market to reach and test more people.

“LOW-COST HEARING TESTING SYSTEM AND METHOD OF COLLECTING USER INFORMATION”, International Application PCT/US2005/______, filed Jun. 10, 2005, claiming priority of U.S. Provisional Application No. 60/579,369, filed Jun. 14, 2004, incorporated by reference herein and assigned to the assignee of this application, describes a hearing testing system that can be administered through a standard low-cost data storage media, such as a CD, that is easily mass-marketed as a give-away and is easily used by the mass market. However, a conventional CD has a 700 MB storage limit, thereby restricting the complexity of the hearing test that can be administered through it. Moreover, mechanisms to further broaden the market reach of the hearing test are highly beneficial, as the easier it is for an individual to access a hearing test, the higher the likelihood that he or she will take the test. Therefore, what is needed is an improved way of conducting hearing tests that may be more complex than hearing tests that are administered through a CD, and that can be easily accessible to the mass market of individuals in order to reach and test more people.

Another problem with current methods for testing hearing loss is the inability to store user-specific information in a database and provide clear step-by-step guidance on the actions needed to find a solution once a hearing loss problem is detected. In the case of the Internet hearing test Web site previously described, the results of the test are not directed to another step, nor are they available to another entity, i.e., an audiologist. Therefore, an audiologist must retest the same frequencies and re-question the patient. Thus, there exists a need to streamline the testing process so that low-cost non-professional hearing tests lead to a more professional hearing test.

Another problem with both conventional non-professional hearing tests and the audiologist-administered professional hearing test is that the tests are simple frequency versus amplitude tests and do not take into account speech intelligibility issues. For example, even though an individual may have some hearing loss, he or she may be able to function quite normally, whereas others may have limitations in understanding certain spoken words. Thus, there exists a need to address some of these speech intelligibility issues.

Another problem with current testing methods is that the individual being tested has no idea at the hearing test what having a hearing aid would do to improve his or her quality of life. That is, even if the patient in either the non-professional test or the professional test recognizes hearing loss, the patient has no idea what the improvement would be if a corrective hearing aid were used. Thus, the motivation to get the problem fixed is much less than if the individual could experience the benefits of correction at the time of the test.

### SUMMARY OF THE INVENTION

It is therefore an object of this invention to find a way for the mass market of individuals with potential hearing loss to recognize early onset of hearing loss without the need to visit an audiologist.

Another object of this invention is to develop an improved way of conducting hearing tests that may be more complex than hearing tests that are administered through a CD, and that can be easily accessible to the mass market of individuals in order to reach and test more people.

Another object of this invention is to streamline the testing process so that low-cost non-professional hearing tests lead to a more professional hearing test.

Another object of this invention is to address speech intelligibility issues at some level in hearing aid tests.

Another object of this invention is to show patients what the result of having a hearing aid would do to improve their quality of life, in order to improve the patients' motivation to fix the problem.

It is another object of the present invention to provide step-by-step guidance on the next steps to be taken once a hearing loss is detected.

The present invention provides for a hearing test stored on a centrally located computer that is accessible either by placing a toll-free call through a telephone using the telephone's microphone and/or keypad as an input device, or by establishing an Internet connection to a centrally located computer through a Web site. Both access methods are easily mass-market-able. In the present invention, the user can be led to the system by advertisement or by a low-cost CD hearing test system. This would allow the mass market of individuals with potential hearing loss to recognize early onset of hearing loss without the need to visit the audiologist. The present invention streamlines and connects low-cost, non-professional hearing tests to a more professional hearing test by providing the results of the non-professional hearing test to the user as a code that can be quickly identified by a professional, e.g., an audiologist. The invention also provides testing of the speech intelligibility issues in a hearing aid test, where such tests are administered around words, based upon the specific results of the hearing test.

The present invention also provides a means to show the user what having a hearing aid would do to improve quality of life by having the system play corrected words or sounds based upon the hearing loss detected. Experiencing such correction in an immediate fashion should improve the patient's motivation to fix the problem. The present invention provides step-by-step guidance on the next steps to be taken if hearing loss is found. Further, this invention also provides a means to store and organize the user test data to create a means for reuse of the data.

In a preferred embodiment, the present invention provides for a remotely accessible data storage media for use in testing hearing of an individual. The media comprises a plurality of hearing test queries, such as a frequency tone, word or sentence, and instruction data. The instruction data includes instructions for automatically or manually operating a local hearing test unit to perform a hearing test based on the hearing test queries retrieved from the media. The hearing test queries include at least one of a set of frequency versus amplitude hearing test and speech intelligibility queries, where the speech intelligibility queries are selectably accessible. The instruction data includes instructions (i) linked to at least one of the frequency versus amplitude queries and (ii) identifying selected user inputs associated with results of the frequency versus amplitude queries and corresponding to selected ones of the speech queries.

In a further embodiment, the media includes a memory for storing user information and user hearing test results obtained from performing the hearing aid testing with the test queries contained on the media, where the user information and the user hearing test results are generated at the hearing test unit.

In another embodiment, the media includes incentive data (e.g. electronic coupons) linked to selected ones of the instruction data. The instruction data, for example, indicate the end of a hearing test, or constitute a code corresponding to the hearing loss profile for the user obtained based on the results of a preliminary hearing test.

In a further embodiment, the media includes hearing test queries indexed with a hearing test code, where the code represents the results of a preliminary hearing test on the individual.

In a further embodiment, the media includes normal and modified word units having the same index as a hearing test code or one of the selected user inputs identified in the instruction data.

The present invention further provides a system for performing a hearing test including a central controller and a hearing test unit. The controller is coupled to the inventive data storage media, and the controller and media are located remotely from the hearing test unit. Each of the controller and the hearing test unit includes means for providing data communications over a data communications network. At least one of the controller and the media include a memory for storing user information and user hearing test results obtained from performing the hearing aid testing with the test queries of the media, where the user information and the user hearing test results are generated at the hearing test unit.

### BRIEF DESCRIPTION OF THE DRAWINGS

Other objects and advantages of the present invention will be apparent from the following detailed description of the presently preferred embodiments, which description should be considered in conjunction with the accompanying drawings in which like references indicate similar elements and in which:

FIG. 1 is a high-level system diagram of an automated and convenient hearing testing system that collects and stores user information.

FIGS. 2, 3 and 4 illustrate methods of using an automated and convenient pre-test hearing testing system that collects and stores user information.

FIG. 5 is a frequency vs. amplitude address lookup table.

FIGS. 6A and 6B illustrate a hearing test questionnaire.

### DETAILED DESCRIPTION OF THE INVENTION

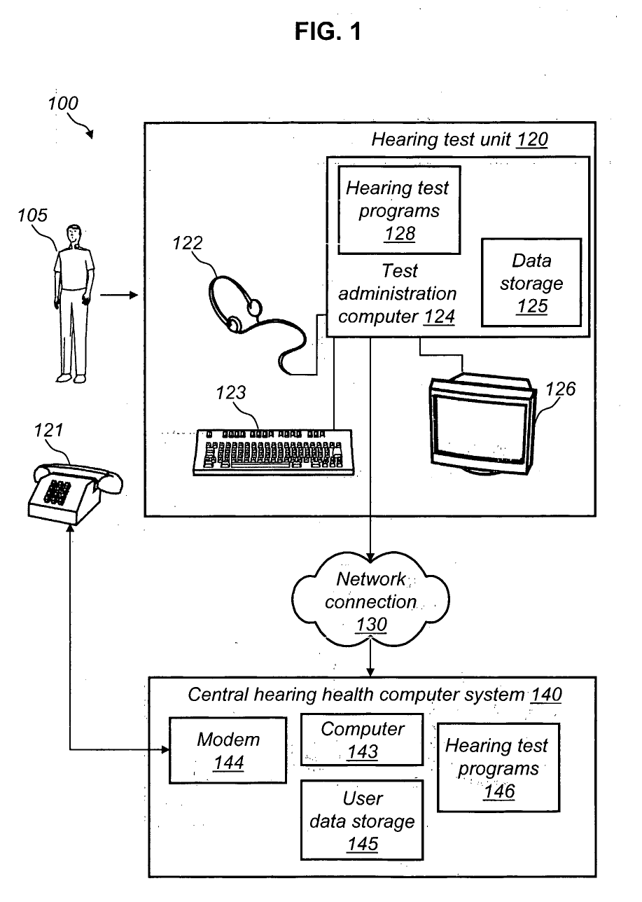

FIG. 1 is a high-level diagram of a preferred system 100 including a user 105, a hearing test unit 120, a network connection 130, a central hearing health computer system 140, and a telephone 121.

User 105 represents the individuals (mass market) on whom a hearing test is to be administered. This is generally any and all individuals but, more specifically, the more than 10% of the population (e.g., 25 million Americans) that have hearing loss, including one out of four people older than 65.

Network connection 130 is a standard Internet connection or, alternatively, is a WAN, LAN, etc. Network connection 130 is the communication infrastructure between hearing test unit 120 and central hearing health computer system 140. Network connection 130 allows central hearing health computer system 140 to remotely administer hearing aid tests, thereby giving central hearing health computer system 140 an opportunity to reach a large number of individuals.

Telephone 121 is a standard telephone capable of generating tone pulse from its keypad. Telephone 121 functions as an input/output device that allow user 105 to communicate with central hearing health computer system 140.

Central hearing health computer system 140 further includes a computer 143, a user data storage 145, a modem 144, and a series of hearing test programs 146.

Central hearing health computer system 140 is a centrally located computer system that is connected to an Internet. Central hearing health computer system 140 is a central repository of all current audiological programs, audiological data, audiological research, sound “.wav” files, and speech and other sound simulations files. Central hearing health computer system 140 centralizes information so that all connected audiologists around the world can access current audiological test procedures, new standards, new algorithms for programming the DSP-based hearing aids, etc.

User data storage 145 is a memory region of central hearing health computer system 140 that stores data concerning user 105 including information such as demographics, age, name, date of birth, etc., and also includes user 105's actual responses to the hearing tests.

Computer 143 is a computer that is capable of performing all conventional computer functions of reading and writing data to memory (within computer 143), reading and writing data to other connected computers, communicating through modem 144 or network connection 130, and running hearing test programs 146.

Hearing test programs 146 include the programs that execute the methods of the present invention.

Hearing test unit 120 further includes a test administrator computer 124, a pair of headphones 122, a keyboard 123, a monitor 126, a data storage 125, and a series of hearing test programs 128.

Hearing test programs 128 running on test administrator computer 124 perform the steps of uploading and running the current hearing test programs 146 on central hearing health computer system 140 to the memory (not shown) of test administrator computer 124, and then downloading the obtained test results data to data storage 125 and central hearing health computer system 140.

User 105 is an individual that tests his or her hearing to determine hearing loss. Test administrator computer 124 is essentially an automatic audiometer that is easily operable by user 105, i.e., it does not need additional supervision. Automatic audiometers are well known in the prior art and any type can work with this invention. For example, U.S. Pat. No. 4,107,465, “Automatic audiometer system,” assigned to Centre de Recherche Industrielle du Quebec, describes an audiometer for testing the hearing characteristic of a person. The audiometer is entirely operable by the person, whereby technicians are not required. The audiometer comprises a source of audible and selectable fixed frequency signals. An automatic frequency selector switch selects a predetermined frequency signal from the source. A variable attenuator circuit is provided to automatically attenuate, in sequence, the predetermined frequency through a plurality of attenuation levels and according to a pre-selected mode of operation whereby to transmit a plurality of attenuated frequency signals. The person using the audiometer transmits the attenuated frequency signals for audible reception. Visual display lamps indicate the test frequencies and attenuation, permitting the person to fill out a test chart on corresponding sounds audible to his or her ears. A control circuit is provided to enable the frequency selector means and the variable attenuator in accordance with a pre-selected mode of operation.

Central hearing health computer system 140 uses computer 143 to communicate with hearing test unit 120 through network connection 130. Central hearing health computer system 140 also operates user data storage 145, which is a central database repository of information (which can later be reused) about user 105. Depending on the specific application of this invention, data storage 125 and user data storage 145 can be mirror images of each other. User data storage 145 can also have user profile and hearing test information from prior tests, which can be updated to data storage 125 as required.

In a first method of operation of system 100, user 105 can take a hearing test in one of two ways. First, user 105 could dial the toll-free telephone number given to him or her at a previous low-cost hearing test. The number dialed links user 105 to central hearing health computer system 140 through modem 144. Computer 143, recognizing the input from modem 144, runs hearing test programs 146 and any data collected is stored in user data storage 145. Hearing test programs 146 can be run in many ways, preferably where the hearing test program 146 sends sounds (tones) at various amplitudes and prompts user 105 to interact through either verbal or keypad responses. In addition, speech intelligibility can be tested by a program with pre-defined sentences that are output to user 105 for his or her understanding and response. In this way, user 105 can take low-cost, non-professional hearing tests at home. Even though it is understood that the telephone system has low bandwidth capability, some amount of useful testing can be done; digital telephone systems are also improving, adding higher frequency capability. It is further understood that, if user 105 had a code from a low-cost test that he or she had previously taken, the first request of the program would be for user 105 to enter the code using the telephone keypad.

A second, improved method of operation to take a hearing test is for user 105 to take the test on hearing test unit 120. This device is a low-cost device that could be available for use at general practitioners' offices or other public areas, such as kiosks in shopping malls, eyeglass shops, or any other similar public area where it would make sense for user 105 to take a hearing test. In this method of operation, user 105 initializes hearing test unit 120, which in turn uploads the current hearing test program 146 on central hearing health computer system 140 through network connection 130. Hearing test unit 120 stores the program as the current hearing test program 128. By using test administrator computer 124, headphones 122, keyboard 123, and monitor 126, user 105 interacts with system 100 in a similar manner as in first method of operation above. The program can be run in many ways, preferably where the hearing test program 128 sends sounds (tones) at various amplitudes and prompts user 105 to interact through either verbal or keypad responses. In addition, speech intelligibility can be tested by a program with pre-defined sentences that are output to user 105 for his or her understanding and response. In this way, user 105 can take low-cost, non-professional hearing tests in a variety of convenient settings. This is an improved system since hearing test unit 120 has higher bandwidth capability than the telephone system. It is further understood that, if user 105 had a code from a low-cost test that he or she had previously taken, the first request of the program would be for user 105 to enter the code using keyboard 123.

There are several alternative ways of reusing user 105 test results and/or profile data that is stored in data storage 125 and user data storage 145. For example, central hearing health computer system 140 can provide user 105 test data to better guide a physician to conduct a more detailed and thorough audiometric test on user 105 and to make recommendations on a remedy in case of hearing loss. User 105 test data from multiple users can also be used for due diligence and statistical analysis to determine preferences for certain users with specific profiles. This can allow for improved targeted marketing of hearing health products to the users.

System 100 is an automated and convenient pre-professional test hearing testing system that collects and stores user information.

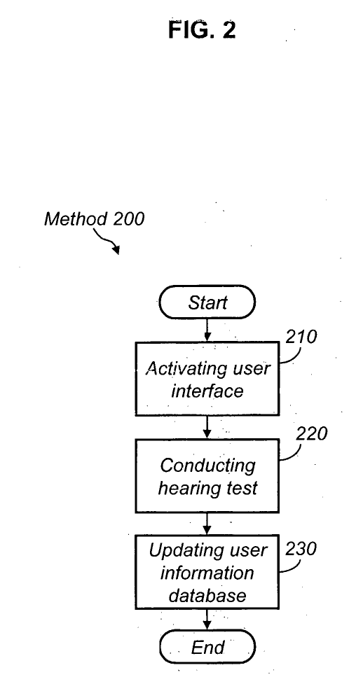

FIG. 2 illustrates a method 200 of using system 100, including the steps of:

Step 210: Activating User Interface

In this step, user 105 activates test administration computer 124, which can be found at general practitioners' offices or other public areas, such as kiosks in shopping malls, or any other similar public area where it would make sense for user 105 to take a hearing test.

Alternatively, user 105 can call into central hearing health computer system 140 from a remote location by using telephone 121. However, current telephone technology limits the maximum test frequency, which can lead to a limited test level for user 105. More so, telephones differ widely across levels of audibility, given that volume settings on telephones are highly variable. This further makes the use of current telephone technology limiting for this invention. As telephone technology improves, the use of telephone 121 to remotely conduct a hearing test can become more popular.

Step 220: Conducting Hearing Test

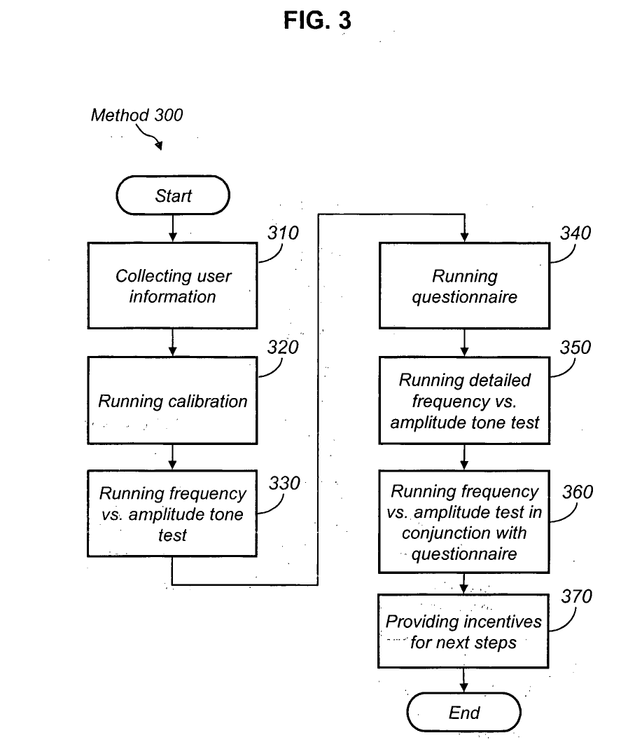

In this step, user 105 takes a hearing test using either test administration computer 124 or central hearing health computer system 140. One mode of operation is explained in the method described with reference to FIG. 3.

Step 230: Updating User Information Database

In this step, user data storage 145 is updated with user 105's hearing test results either directly, if user 105 connected through telephone 121, or indirectly when data storage 125 connects with user data storage 145 through network connection 130 and updates or adds the collected information from user 105. Method 200 ends.

FIG. 3 illustrates a method 300 of conducting a hearing test using system 100. It is assumed that user 105 has already connected either by using telephone 121 to connect to central hearing health computer system 140 directly or by using test administration computer 124. Method 300 includes the steps of:

Step 310: Collecting User Information

In this step, user 105 enters his or her personal profile and contact information by using either keyboard 123 or the keypad on telephone 121, and the information is stored on data storage 125. Note that before collecting information, user 105 is notified that his/her information will be strictly kept as private and secure from unwanted third parties. Other conventional voice response technology can also be used in place of the keypad for telephone 121. If user 105 has received a code when taking a previous low-cost hearing test, the code is entered at this time. This code refers to a lookup table on computer 143 or test administration computer 124 to determine the results of the earlier test and is used to improve this hearing aid test.

Step 320: Running Calibration

In this step, user 105 calibrates and initiates hearing test program 128 or hearing test program 146, which provides a verbal set of directions that tells user 105 to listen to the following tone and to set the volume (e.g., via keyboard 123) to its lowest audible level. Setting the volume to its lowest audible level is an optimal environment to conduct an accurate hearing test. User 105 is then guided to the first frequency test module that is based upon the correct volume level set by the user.

Step 330: Running Frequency Vs. Amplitude Tone Test

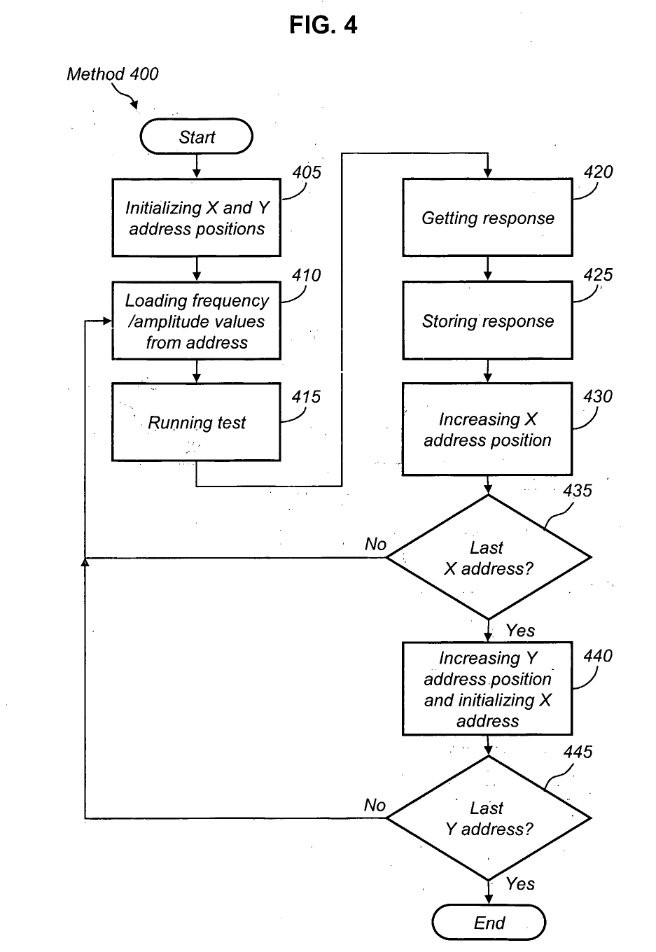

In this step, user 105 uses hearing test program 128 or hearing test program 146 to conduct a frequency vs. amplitude tone test. A frequency vs. amplitude tone test is detailed below in reference to FIG. 4; however, such tests are well known in the art and this invention only illustrates a simplified method.

Step 340: Running Questionnaire

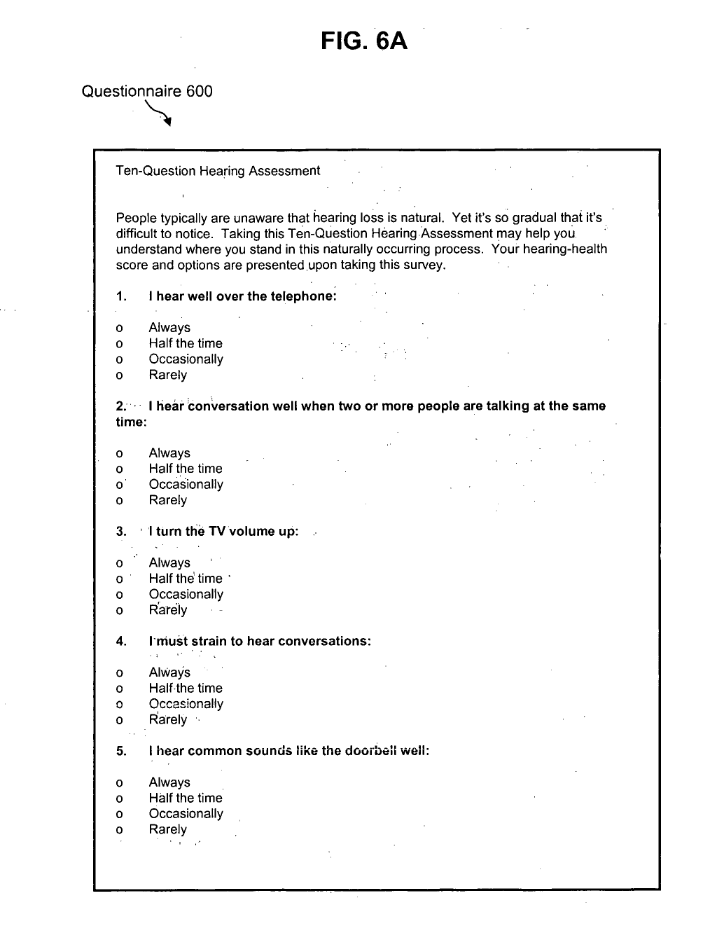

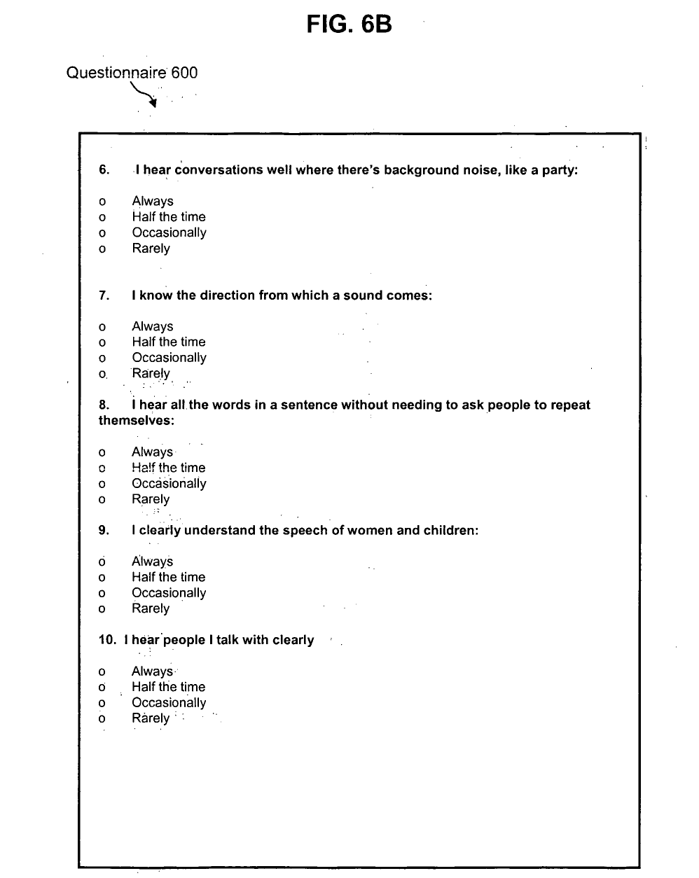

In this step, hearing test program 128 or hearing test program 146 conducts a questionnaire 600 (shown in FIGS. 6A and 6B). By answering questionnaire 600, user 105 can further confirm his or her level of hearing loss. The questions on questionnaire 600 can be easily modified according to the specific responses given by user 105 to hearing test program 128 or hearing test program 146, and questions well known in the art can also be added.

Step 350: Running Detailed Frequency Vs. Amplitude Tone Test

In this step, hearing test program 128 or hearing test program 146 conducts a detailed frequency vs. amplitude test of user 105. This test is usually conducted to isolate and confirm user 105's deficiency type. Detailed frequency vs. amplitude tone tests are well known in the art.

Step 360: Running Frequency Vs. Amplitude Test in Conjunction with Questionnaire

In this step, hearing test program 128 or hearing test program 146 conducts a frequency vs. amplitude test on user 105 in conjunction with his or her responses from questionnaire 600. For example, if user 105 has answered on questionnaire 600 that background noise affects him or her, a more detailed frequency vs. amplitude test can be run, with and without background noise, to determine the effects. Running detailed frequency vs. amplitude tone tests in conjunction with questionnaires is well known in the art.

Step 370: Providing Incentives for Next Steps

In this step, if it has been determined that user 105 has a hearing loss, an added incentive to begin corrective measures can be provided to user 105. For example, a $100 cost savings “coupon” on hearing aids can be provided as an incentive. Methods of printing and providing physical coupons, or providing electronic coupons via the Internet, are known in the art. Method 300 ends:

In addition in step 370, following testing, the hearing test unit 120 executes a hearing test improvement demonstration program, which is stored in either the hearing test programs 126 or 146, to provide that hearing aid corrected (modified) words and normal words are played for the individual. The modified words are amplified versions of the normal words which are used to demonstrate the improvement in the individual's hearing that would be achieved through use of a hearing aid, whereas the normal words do not include any amplifications.

FIG. 4 illustrates a method 400 of conducting a frequency vs. amplitude tone test, including the steps of:

Step 405: Initializing X and Y Address Positions

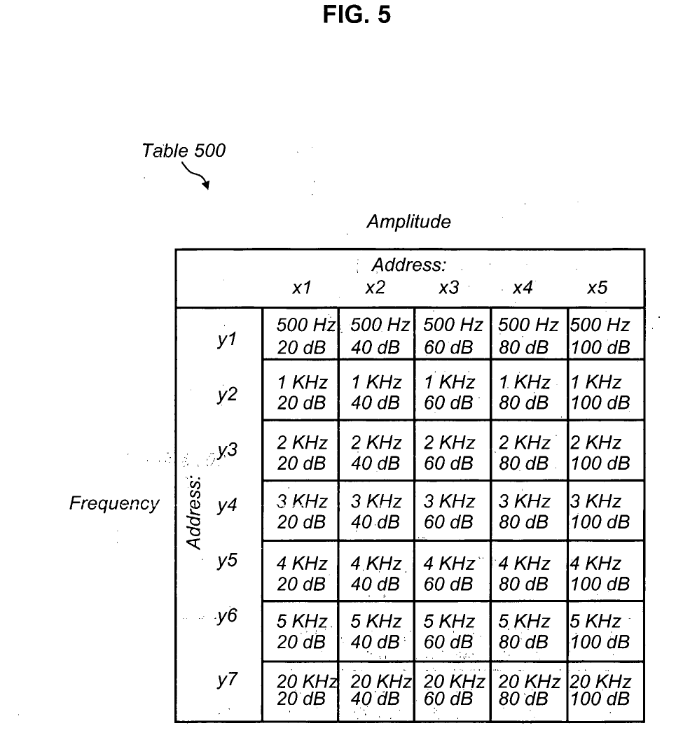

In this step, hearing test program 128 or hearing test program 146 initiates the X and Y address positions of amplitude and frequency values to their starting positions. Values for the X and Y positions are shown in FIG. 5, which illustrates a frequency vs. amplitude lookup table 500 that is used to administer the frequency vs. amplitude tone test on user 105. Table 500 is divided into X addresses X1-X5 that represent increments in amplitude, and Y addresses Y1-Y5 that represent increments in frequency.

Step 410: Loading Frequency/Amplitude Values from Address

In this step, the frequency and amplitude values corresponding to the X and Y address locations in table 500 are loaded into a frequency-amplitude player such as an amplifier.

Step 415: Running Test

In this step, the frequency and amplitude values are played in headphones 122. Alternatively, if user 105 is calling through telephone 121, the frequency and amplitude values are played in through telephone 121.

Step 420: Getting Response

In this step, user 105 is asked a question such as whether the frequency and amplitude values are audible. User 105 responds to the question accordingly through keyboard 123 or telephone 121.

Step 425: Storing Response

In this step, user 105's response to the question asked in step 420 is stored in data storage 125 or user data storage 145. Central hearing health computer system 140 can later reuse this response.

Step 430: Increasing X Address Position

In this step, the X address location in table 500 is increased.

Step 435: Last X Address?

In this decision step, the program determines whether the last X address has been reached. If so, method 400 proceeds to step 440; if not, method 400 returns to step 410.

Step 440: Increasing Y Address Position and Initializing X Address

In this step, the Y address location in table 500 is increased and the X address location is initialized to its starting location.

Step 445: Last Y Address?

In this decision step, the program determines whether the last Y address has been reached. If so, method 400 ends; if not, method 400 returns to step 410.

FIGS. 6A and 6B illustrate questionnaire 600 that was referenced in method 300.

Thus, the invention provides for the collection and storage of user information, via an automated and convenient pre-professional test hearing testing system, in a database that can be later accessed to allow for reuse of the information. Those of ordinary skill in the art will realize that the above description of a hearing testing system is illustrative only and not in any way limiting. Other embodiments of a hearing testing system will readily suggest themselves to such skilled persons.

Although preferred embodiments of the present invention have been described and illustrated, it will be apparent to those skilled in the art that various modifications may be made without departing from the principles of the invention.

What is claimed is:

1. A remotely accessible data storage media for use in testing hearing of an individual comprising:
a plurality of hearing test queries and instruction data, wherein the instruction data includes instructions for operating a local hearing test unit to perform a hearing test using the hearing test queries on the media,
wherein the hearing test queries include at least one of a set of frequency versus amplitude hearing test and speech intelligibility queries, wherein the speech intelligibility queries are selectably accessible, and
wherein the instruction data includes instructions linked to at least one of the frequency versus amplitude queries and identifying selected user inputs associated with results of the frequency versus amplitude queries and corresponding to selected ones of the speech queries.

2. The media of claim 1 further comprising:
a memory for storing user information and user hearing test results obtained from performing the hearing testing with the test queries of the media, wherein the user information and the user hearing test results are generated at the hearing test unit.

3. The media of claim 1 further comprising:
incentive data linked to selected ones of the instruction data.

4. The media of claim 3, wherein the incentive data includes an electronic coupon.

5. The media of claim 3, wherein the instruction data indicate an end of a hearing test or a code corresponding to a hearing loss profile for the user obtained based on the results of a preliminary hearing test.

6. The media of claim 1, wherein at least one of the hearing test queries is indexed with a hearing test code, wherein the code represents the results of a preliminary hearing test on the individual.

7. The media of claim 6 further comprising:
normal and modified word units having the same index as one of the hearing test codes or one of the selected user inputs identified in the instruction data.

8. A system for performing a hearing test including:
a central controller and a hearing test unit, wherein the controller is coupled to a remotely accessible data storage media and wherein the controller and media are located remotely from the hearing test unit;
wherein the media comprises:
a plurality of hearing test queries and instruction data, wherein the instruction data includes instructions for operating the hearing test unit to perform a hearing test using the hearing test queries on the media,
wherein the hearing test queries include at least one of a set of frequency versus amplitude hearing test and speech intelligibility queries, wherein the speech intelligibility queries are selectably accessible, and
wherein the instruction data includes instructions linked to at least one of the frequency versus amplitude queries and identifying selected user inputs associated with results of the frequency versus amplitude queries and corresponding to selected ones of the speech queries; and
wherein each of the controller and the hearing test unit includes means for providing data communications over a data communications network.

9. The system of claim 8, wherein at least one of the controller and the media includes a memory for storing user information and user hearing test results obtained from performing the hearing test with the test queries of the media for a plurality of individuals, wherein the hearing test results are remotely accessible.

10. A method for performing a hearing test comprising:
providing a local hearing test unit;
providing a remotely accessible data storage media for use by the local hearing test unit in testing hearing of an individual, wherein the media comprises:
a plurality of hearing test queries and instruction data, wherein the instruction data includes instructions for operating the local hearing test unit to perform a hearing test using the hearing test queries on the media,
wherein the hearing test queries include at least one of a set of frequency versus amplitude hearing test and speech intelligibility queries, wherein the speech intelligibility queries are selectably accessible, and
wherein the instruction data includes instructions linked to at least one of the frequency versus amplitude queries and identifying selected user inputs associated with results of the frequency versus amplitude queries and corresponding to selected ones of the speech queries; and
providing a remotely accessible memory for storing user information and user hearing test results obtained from performing the hearing test with the test queries of the media, wherein the user information and the user hearing test results are generated at the hearing test unit.
